(19) United States
(12) Patent Application Publication
(10) Pub. No.: US 2016/0168534 A1
(43) Pub. Date: Jun. 16, 2016

(54) Methods of Embryogenic Tissue Preparation for Sugar Cane Transformation

(71) Applicant: Syngenta Participations AG, Basel (CH)

(72) Inventors: Pearl Mai, Research Triangle Park, NC (US); Erik Dunder, Research Triangle Park, NC (US)

(21) Appl. No.: 14/569,254

(22) Filed: Dec. 12, 2014

(51) Int. Cl. C12N 5/04

(52) U.S. Cl. CPC C12N 5/04

(57) ABSTRACT

Methods of tissue preparation for the transformation of sugar cane are provided. The methods comprise a pre-culture treatment of sugar cane embryogenic tissue prior to transformation. The methods comprise excising a segment of plant tissue from a shoot of sugar cane; culturing said segment to produce sugar cane embryogenic tissues; performing a pre-culturing treatment by sub-culturing responding embryogenic tissue on fresh media for a period of time of at least 7 days, in the same media; and with no intervening step and no changes of media, performing transformation of embryogenic tissue. Transformation can be performed via Agrobacterium-mediated gene delivery, biolistic transformation, and the like. Transgenic plants are regenerated from plantlets grown under conditions favoring growth of transformed cells while substantially inhibiting growth of non-transformed cells.

### FIELD OF THE INVENTION

This invention relates to plant molecular biology, particularly to methods and compositions for preparation of sugar cane embryogenic tissues for the transformation of sugar cane.

### BACKGROUND OF THE INVENTION

Sugar cane (Saccharum spp.) is a highly polyploid plant grown in different parts of the world from the tropics to the subtropics, and accounts for around 60% of the world's sugar. It is also one of the important cash crops in many countries, with a high trade value. The importance of sugar cane has increased in recent years because cane is an important raw material for sugar industries and allied industries producing alcohol, acetic acid, butanol, paper, plywood, industrial enzymes and animal feed. Considering its importance in the agricultural industry, concerted efforts are being made for its improvement using biotechnological approaches.

The importance of sugar cane transformation is increasing as a means to introduce useful and improved traits into many cultivars of economical relevance for integrated crop management and biofuels applications. Some of the main traits to be improved by genetic engineering are: tolerances to viruses, insects, and fungus attacks, herbicide resistance, improvement of the fiber quality and the use of sugar cane plants as bioreactors.

Although Agrobacterium-mediated transformation has been used for genetic manipulation of sugar cane, efficiency and reproducibility of the available methodologies continue to be a challenge. In fact, Agrobacterium tumefaciens induces necrosis in cultured, transformed sugar cane tissue, with a resultant low transformation frequency (Arencibia et al. (1998) Transgenic Res. 7:123-222; Enriquez-Obregon et al. (1997) Biotecnologia Aplicada 14:169-174; and de la Riva et al. (1998) Electron. J. Biotechno. 1:118-133).

Because of the importance of manipulating sugar cane for improved characteristics, there is a need for additional methods that advantageously increase the efficiency of Agrobacterium-mediated transformation of this important agricultural crop. Accordingly, the present invention overcomes the deficiencies in the art by providing methods of preparation of the target tissue prior to transformation that result in greater transformation efficiencies. It is well-accepted in the art to culture embryogenic cultures on fresh media for a few days before transformation. This relatively short time is believed to maximize the amount of actively dividing cells, which are the cells most amenable to transformation. The methods described here teach increasing the amount of time embryogenic cultures are cultured on a solid media prior to transformation, approaching potential nutrient exhaustion, and that doing so surprisingly increases transformation efficiency.

### SUMMARY OF THE INVENTION

Methods of preparing sugar cane embryogenic tissue prior to transformation by performing a pre-treatment of embryogenic cultures are provided. The methods comprise excising a segment of plant tissue from a shoot of sugar cane; culturing said segment to produce embryogenic tissues; and performing a pre-culturing treatment by sub-culturing responding sugar cane embryogenic tissues to fresh media and allowing it to grow for a period of time of at least 7 days, on the same culture media, to further develop the embryogenic tissues, wherein sugar cane embryogenic tissues responding to the pre-culture treatment are more transformable. In some embodiments, the pre-culturing treatment prior to transformation is for a period of time of at least 7 days. In some embodiments, the pre-culturing treatment prior to transformation is for a period of time of 7 to 60 days. In some embodiments, the pre-culturing treatment prior to transformation is for a period of time of 7 to 50 days. In some embodiments, the pre-culturing treatment prior to transformation is for a period of time of 7 to 40 days. In some embodiments, the pre-culturing treatment prior to transformation is for a period of time of 7 to 35 days. In some embodiments, the pre-culturing treatment prior to transformation is for a period of time of 7 to 30 days. In some embodiments, the pre-culturing treatment prior to transformation is for a period of time of 7 to 25 days. In some embodiments, the pre-culturing treatment prior to transformation is for a period of time of 7 to 21 days. In some embodiments, the pre-culturing treatment prior to transformation is for a period of time of 7 to 14 days. In some embodiments, the method of transformation following the pre-culturing treatment is Agrobacterium-mediated. In some embodiments, the sugar cane embryogenic tissues are removed from the media following the pre-culture treatment and mixed with a solution comprising Agrobacterium in suspension as part of the transformation process.

In some embodiments, the pre-culture treatment of embryogenic tissues occurs after a first step of culturing embryogenic cultures. In some embodiments, the method comprises excising a segment of plant tissue from a shoot of sugar cane; culturing said segment of plant tissue to produce sugar cane embryogenic cultures; sub-culturing responding sugar cane cultures on fresh media; optionally repeating said for at least 7 days; and performing a pre-culturing treatment comprising responding sugar cane tissues to fresh media and culturing for a period of time of at least 7 days in the same media, wherein sugar cane embryogenic tissues responding to the pre-culture treatment are transformable. In some embodiments, the pre-culturing treatment prior to transformation is for a period of time of at least 7 days. In some embodiments, the pre-culturing treatment prior to transformation is for a period of time of 7 to 60 days. In some embodiments, the pre-culturing treatment prior to transformation is for a period of time of 7 to 50 days. In some embodiments, the pre-culturing treatment prior to transformation is for a period of time of 7 to 40 days. In some embodiments, the pre-culturing treatment prior to transformation is for a period of time of 7 to 35 days. In some embodiments, the pre-culturing treatment prior to transformation is for a period of time of 7 to 30 days. In some embodiments, the pre-culturing treatment prior to transformation is for a period of time of 7 to 25 days. In some embodiments, the pre-culturing treatment prior to transformation is for a period of time of 7 to 21 days. In some embodiments, the pre-culturing treatment prior to transformation is for a period of time of 7 to 14 days. In some embodiments, the method of transformation following the pre-culturing treatment is Agrobacterium-mediated. In some embodiments, the sugar cane embryogenic cultures are removed from the media following the pre-culture treatment and mixed with a solution comprising Agrobacterium in suspension as part of the transformation process.

Overview

Successful sugar cane transformation has been reported using both particle- and Agrobacterium-mediated gene delivery methods. Agrobacterium-mediated transformation is a preferred method, due to potential advantages of simpler methodology and a higher proportion of simple transgene integration events. Because monocotyledonous species such as grasses are not natural hosts of Agrobacterium, conditions must be determined for the best transformation efficiency. It is generally accepted in the art that an important factor in Agrobacterium-mediated transformation is the inoculation of actively dividing cells, typically in immature embryos or embryogenic cultures. Therefore, tissue is typically prepared in a manner to encourage the growth of actively dividing cells. This includes maintaining the tissue on nutrient-rich media supplemented with phytohormones to promote tissue growth, and replenishing said media before any nutrients or carbohydrates are exhausted. It is generally accepted in the art that exhaustion of the media would result in a decrease of rapid cell growth and a subsequent decrease in the number of actively dividing cells. This decrease would decrease the number of cells amenable to Agrobacterium-mediated transformation.

The present invention provides a method of pre-culturing which results in increased transformation efficiency of sugar cane. Surprisingly, the pre-culture treatment requires culturing of embryogenic cultures for an extended period of time on a plate of solid media, such that the nutrients are approaching exhaustion, prior to transformation. The embryogenic cultures develop a more mature and established morphology with this increased preculture time.

The article “a” and “an” are used herein to refer to one or more than one (i.e., to at least one) of the grammatical object of the article. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps.

Explants

The explants described herein are sugar cane embryogenic cultures. For the purposes of the present invention, “sugar cane” will refer to any Saccharum plant or hybrid. Hybrid plants include those generated by the traditional Saccharum spontaneum by Saccharum officianarum hybrid material that makes up all current commercial sugar cane and energycane germplasm, and any other hybrids that are produced by crossing sugar cane with closely or distantly related species. Examples of other species that sugar cane can be crossed with to generate hybrid plants or new varieties of sugar cane include Miscanthus and Sorghum.

For sugar cane transformation, the explants used as targets for transformation are primarily embryogenic cultures derived from young leaf bases from the tops of 3-9 month old field or greenhouse grown plants or immature flowers of sugar cane plants.

The tissue above the apical meristem that is excised from the immature or mature shoot can be cultured directly. Alternatively, the tissue can be cut up in many different ways, such as cut longitudinally, cut horizontally, or cut into segments, to generate many explants as source material for culture. The explant may be obtained from immature shoot tissues including leaf spindle or whorl, stems, leaf sheath, leaf roll (meristematic region), node, or internode segments. In some embodiments, the explant is leaf sheath or leaf roll sections. The segment may be cut from just above the apical meristem up to about 3 cm or up to about 6 cm above the apical meristem. In various embodiments, the explant is not a node segment or is not an internode segment. As used in the context of the invention, the term “node segment” includes any joint in a stem from where one or more leaves may grow and also includes any lateral (axillary) buds on the side of the stem, as in a leaf axil. The part of the stem between two nodes is termed the “internode.” The outer one or two leaves may be removed from the immature shoot prior to segmenting.

The variety of different explants that can be excised from the immature shoot can be placed into in vitro culture for some hours, days, weeks or months prior to targeting them for gene delivery. This culture period can produce an embryogenic culture line or/and callus that can also serve as a target for transformation.

Embryogenic Culture Induction

In some embodiments, the cut segments are cultured under conditions sufficient to induce embryogenic culture response and produce embryogenic cultures. The term “callus” or “calli” refer to an undifferentiated proliferating mass of cells or tissue. “Embryogenic cultures” comprise embryogenic tissues, and are characterized by a proliferation of somatic embryos in vitro. At the start of the in vitro culture process, embryogenesis is induced from the vascular meristems in immature leaf tissue. The tissue responding to the embryogenesis induction treatment is typically transferred onto fresh media with or without selection, a step referred to as “sub-culturing.” Sub-culturing may also refer to selecting the best quality embryogenic tissue from an embryogenic culture and placing it on fresh media for further growth. This culture then undergoes secondary embryogenesis to produce successive generations of somatic embryos. Since somatic embryos have a single cell origin, this culture is an excellent target for transformation to quickly produce completely transgenic embryos derived from individual transgene integration events.

Culture conditions sufficient for embryogenic culture formation are known to those skilled in the art, and may vary according to sugar cane cultivar. Suitable media for establishment and maintenance of embryogenic cultures are described in, e. g. Wang ed. Methods in Molecular Biology Vol. 344, page 227-235; Published International Application No. WO 01/33943, U.S. Pat. No. 5,908,771, U.S. Pat. No. 6,242,257, Croy (Ed.) Plant Molecular Biology Labfax, Bios Scientific Publishers Ltd. (1993), Jones (Ed.) Plant Transfer and Expression Protocols, Humana Press (1995), and in the references cited therein. Each of these references is incorporated herein by reference in their entirety. Additional details relating to culturing plant cells, including pretreatment processes, are provided below in the examples.

The explant may be cultured from about 0 to about 90 days, inclusive, prior to transformation. In various embodiments, the explant is cultured for about 5 days, about 6 days, about 7 days, about 8, about 9, about 10, about 12, about 14, about 16, about 20, about 25, about 30, about 40, about 50, about 60, about 70, about 80 or about 90 days prior to transformation. The culture medium may include Murashige & Skoog (MS) nutrient formulation (Murashige & Skoog, 1962, Physiologia Plantarum 15 473) or Gamborg's medium (Gamborg et al., 1968, Exp. Cell Res 50 15 1). Preferably, the medium comprises MS formulation. It will be appreciated that the above mentioned media are commercially available, as are other potentially useful media.

The medium may further comprise sucrose, and may additionally include agar. Thus, it will be appreciated that the explant may be cultured in solid or liquid medium.

Additional components of the medium may include phytohormones such as cytokinin and/or auxin. In various embodiments, the cytokinin is selected from the group consisting of kinetin, TDZ, and N₆-benzyladenine (BA). There are a variety of other cytokinins, or cytokinin-like, compounds which may be useful according to the present invention, for example zeatin, α-isopentyladenosine, and diphenylurea.

In various embodiments, the auxin is 1-napthaleneacetic acid (NAA) or 2,4 dichlorophenoxyacetic acid (2,4D). There are a variety of other auxins or auxin-like compounds which may be useful according to the present invention, for example dicamba, indole-3-butyric acid (IBA), p-chlorophenoxyacetic acid (CPA), indole-3-acetic acid (IAA), 2,4,5-trichlorophenoxyacetic acid, phenylacetic acid, picloram, β-napthoxyacetic acid, dicamba and trans-cinnamic acid.

It will be readily apparent to the skilled artisan that the most efficacious concentrations of auxin and/or cytokinin can be determined empirically by cross-testing various concentrations of auxin and cytokinin. The optimal concentration of either or both can be tailored according to the particular plant cultivar from which the cultured explant was taken.

Following initial embryogenic culture formation, high quality responses are optionally sub-cultured for about 1 to about 90 days, inclusive, to bulk up the cultures for transformation. Embryogenic cultures are cultures composed of somatic embryos and/or cells that are differentiated to varying degrees. When induced to further differentiate and regenerate, shoots can arise from these cultures by either embryogenesis, organogenesis or some combination of these two processes.

The variety of different explants that can be excised from the immature shoot can serve as either a target for immediate transformation or as a culture source to generate transformation target material. Explants can be immediately targeted for gene delivery by particle delivery, Agrobacterium or other methods of gene delivery. Alternatively, these explants can be placed into in vitro culture for some hours, days, weeks or months prior to targeting them for gene delivery. This culture period can produce an embryogenic culture line or/and callus that can also serve as a target for transformation.

Pre-Culture Treatment

The “pre-culture step” or “pre-culture treatment” is defined as the period of time that the target tissue is cultured on or in the same container of media prior to transformation. The “target tissue” comprises actively dividing cells, or “target cells,” which are known in the art to be the most likely to uptake foreign DNA. The target tissue can be embryogenic cultures comprising embryogenic tissues. During the pre-culture treatment, the target tissue grows, various nutrients are metabolized, the in vitro environment changes, and the target tissue becomes more amenable to transformation. The media used in the pre-culture treatment is similar to the media used in embryogenic culture induction, as described above. The length of the pre-culture treatment can be at least seven days, at least ten days, at least 14 days, 7-90 days, 7-60 days, 7-30 days, 7-25 days, 7-21 days, or 7-14 days.

In some embodiments of the invention, the embryogenic culture is subsequently transformed with one or more nucleotide sequences of interest, or “foreign DNA”. The expression cassette described herein can be introduced into a cell of the embryogenic culture in a number of art-recognized ways. The term “introducing” in the context of a polynucleotide, for example, a nucleotide construct of interest, is intended to mean presenting to the plant the polynucleotide in such a manner that the polynucleotide gains access to the interior of a cell of the plant. Where more than one polynucleotide is to be introduced, these polynucleotides can be assembled as part of a single nucleotide construct, or as separate nucleotide constructs, and can be located on the same or different transformation vectors. Accordingly, these polynucleotides can be introduced into the host cell of interest in a single transformation event, or in separate transformation events. The methods of the invention do not depend on a particular method for introducing one or more polynucleotides into a plant, only that the polynucleotide(s) gains access to the interior of at least one cell of the plant. Methods for introducing polynucleotides into plants are known in the art including, but not limited to, transient transformation methods, stable transformation methods, and virus-mediated methods.

Transformation

“Transient transformation” in the context of a polynucleotide is intended to mean that a polynucleotide is introduced into the plant cell and but has not yet integrated into the genome. Transient expression is a clear indicator of gene delivery efficiency.

By “stably introducing” or “stably introduced” in the context of a polynucleotide introduced into a plant is intended the introduced polynucleotide is stably incorporated into the plant genome, and thus the plant is stably transformed with the polynucleotide.

“Stable transformation” or “stably transformed” is intended to mean that a polynucleotide, for example, a nucleotide construct described herein, is introduced into a plant cell, integrates into the genome of the plant cell, a whole plant is produced from that cell and the integration event is capable of being inherited by the progeny thereof, more particularly, by the progeny of multiple successive generations.

Numerous transformation vectors available for plant transformation are known to those of ordinary skill in the plant transformation arts, and the genes useful to this invention can be used in conjunction with any such vectors. Ti plasmid vectors have been utilized for the delivery of foreign DNA, as well as direct DNA uptake, liposomes, electroporation, microinjection, and microprojectiles. In addition, bacteria from the genus Agrobacterium can be utilized to transform plant cells.

Many vectors are available for transformation using Agrobacterium tumefaciens. These typically carry at least one T-DNA border sequence and include vectors such as pBIN19 (Bevan, Nucl. Acids Res. (1984)). For the construction of vectors useful in Agrobacterium transformation, see, for example, US Patent Application Publication No. 2006/0260011, herein incorporated by reference.

Transformation techniques that do not rely on Agrobacterium include transformation via particle bombardment, protoplast uptake (e.g. PEG and electroporation) and microinjection.

In some embodiments, Agrobacterium-mediated transformation methods are employed. See, WO 94/00977 and U.S. Pat. No. 5,591,616, both of which are incorporated herein by reference. See also, Negrotto et al., Plant Cell Reports 19: 798-803 (2000), incorporated herein by reference. The term “Agrobacterium” refers to species, subspecies, or strains of the bacterium Agrobacterium that are able to mobilize and selectively transfer T-DNA into a plant cell. For example, the Agrobacterium is optionally Agrobacterium rhizogenes, but more typically is Agrobacterium tumefaciens. Suitable Agrobacterium strains include Agrobacterium tumefaciens and Rhizobium rhizogenes (Agrobacterium rhizogenes). While wild-type strains may be used, “disarmed” derivatives of both species, in which the tumor-inducing sequences of the Ti plasmid have been removed, are preferred. Suitable Agrobacterium tumefaciens strains include, e. g., EHA101, as described by Hood et al. ((1986) J. Bacteriol., 168: 1291-1301), LBA4404, as described by Hoekema et al. ((1983) Nature, 303: 179-80), and C58 (pMP90), as described by Koncz and Schell ((1986) Mol. Gen. Genet., 204: 383-96). Preferred Agrobacterium rhizogenes strain are 15834, as described by Birot et al. (Biochem, 25: 323-35) and R1000.

Agrobacterium transformation typically involves the transfer of the binary vector carrying the foreign DNA of interest to an appropriate Agrobacterium strain. Selection of the Agrobacterium strain may depend on the complement of vir genes carried by the host Agrobacterium strain either on a co-resident Ti plasmid or chromosomally (Uknes et al. Plant Cell 5: 159-169 (1993)). The transfer of the recombinant binary vector to Agrobacterium is accomplished by a triparental mating procedure using Escherichia coli carrying the recombinant binary vector, a helper E. coli strain which carries a plasmid and which is able to mobilize the recombinant binary vector to the target Agrobacterium strain. Alternatively, the recombinant binary vector can be transferred to Agrobacterium by DNA transformation (Hofgen & Willmitzer, Nucl. Acids Res. 16: 9877 (1988)).

Transformation of the sugar cane embryogenic tissues by recombinant Agrobacterium usually involves co-cultivation of the Agrobacterium with explants from the plant and follows protocols well known in the art. The embryogenic culture may be removed from the pre-culturing media into a vessel which comprises Agrobacteria suspended in a solution. The co-cultivation of the embryogenic tissues and the Agrobacterium may involve stirring or shaking Transformed tissue is regenerated on selectable medium carrying the antibiotic or selectable marker present between the binary plasmid T-DNA borders.

Selection

After transformation with the nucleotide sequence(s) of interest, the plant material is typically transferred to media that includes a selective agent that is capable of preventing the growth of cells that have not received a gene (e. g., a selectable marker) whose expression product is capable of preventing the action of the selective agent to thereby select for transformed plant cells. The term “selecting” refers to a process in which one or more plants or plant cells are identified as having one or more properties of interest, e. g., a selectable marker, enhanced insect resistance, increased or decreased carotenoid levels, altered coloration, etc. For example, a selection process can include placing organisms under conditions where the growth of those with a particular genotype will be favored.

The selection step comprises culturing the cells that were exposed to the nucleotide sequence of interest under selective conditions. The “selective conditions” include those that are sufficient for distinguishing a transformed cell from a non-transformed cell. Such conditions will vary with, for example, the type of selectable marker used, the cultivar, and the method of transformation, but will generally comprise conditions which favor the growth of transformed cells but inhibit the growth of non-transformed cells.

In certain embodiments, for example, tissues are exposed to sublethal levels of selective agents for about 2-12 weeks, and then to lethal levels of selective agents for about 4-30 weeks in a step-wise selection process. A variety of selectable markers are known in the art. The selectable marker gene may be on the same expression cassette as the nucleotide sequence of interest, or may be contransformed on a separate expression cassette. Selection markers used routinely in transformation include the nptII gene, which confers resistance to kanamycin and related antibiotics (Messing & Vierra. Gene 19: 259-268 (1982); Bevan et al., Nature 304:184-187 (1983)), the bar gene, which confers resistance to the herbicide phosphinothricin (White et al., Nucl. Acids Res 18: 1062 (1990), Spencer et al. Theor. Appl. Genet 79: 625-631 (1990)), the hph gene, which confers resistance to the antibiotic hygromycin (Blochinger & Diggelmann, Mol Cell Biol 4: 2929-2931), and the dhfr gene, which confers resistance to methatrexate (Bourouis et al., EMBO J. 2(7): 1099-1104 (1983)), the EPSPS gene, which confers resistance to glyphosate (U.S. Pat. Nos. 4,940,935 and 5,188,642), and the phosphomannose isomerase gene (PMI), which provides the ability to metabolize mannose (U.S. Pat. Nos. 5,767,378 and 5,994,629). In certain embodiments, cells are transferred to a recovery medium that comprises counter-selective agents (e. g., antibiotics, etc.), e. g., to inhibit the growth of or kill Agrobacterium cells for a period of about 1-15 days, e. g., prior to or concurrently with being transferred to media comprising a selective agent. After a period of culture, plant cells that continue to grow normally are separated from cells whose growth has been slowed or terminated.

Regeneration

Plant tissue growing in the presence of selective agent may be further manipulated for plant regeneration. The term “regenerating” or “generating” refers to the formation of a plant that includes a rooted shoot. The regeneration of plants from various explants is well known in the art. See, e. g., Weissbach et al. (Eds.), Methods for Plant Molecular Biology, Academic Press, Inc. (1988). In certain embodiments of the invention, the regeneration and growth process includes the steps of selecting transformed cells and shoots, rooting the transformed shoots, and growing the plantlets in soil. For example, the regeneration of plants containing a gene introduced by Agrobacterium from leaf explants can be achieved as described by Horsch et al. (1985) Science, 227: 1229-1231. In this procedure, transformants are grown in the presence of a selection agent and in a medium that induces the regeneration of shoots in the plant species being transformed as described by Fraley et al. (1983) Proc. Natl. Acad. Sci. U.S.A., 80: 4803. This procedure typically produces shoots within two to four weeks and these transformed shoots are then transferred to an appropriate root-inducing medium containing the selective agent and an antibiotic to prevent bacterial growth. Typically, transformed shoots that rooted in the presence of the selective agent to form plantlets are then transplanted to soil or other media to allow the production of additional roots.

The transgenic plantlets are then propagated in soil or a soil substitute to promote growth into a mature transgenic plant. Propagation of transgenic plants from these plantlets is performed, for example, in Perlite, peatmoss and sand (1:1:1) under glasshouse conditions.

Detection of Transgene Expression

The above conditions lead to regeneration of green plantlets and plants with photosynthetic ability. A test used for confirmation that the gene is stably integrated into the genome of the host plant necessarily depends on the property to be conferred to the plant. For example, when the property is herbicide resistance, confirmation may be achieved by treatment of the growing plants by spraying or painting the leaves with the herbicide in a concentration that is lethal for control plants that have not been subjected to the transformation process.

### EXPERIMENTAL

### Example 1

### Embryogenic Culture Production

Plant materials were obtained from greenhouse-grown sugar cane plants. Sugar cane varieties examined include a US commercial variety and two Brazilian commercial sugar cane varieties. Plants were grown at the Syngenta Greenhouse complex, Research Triangle Park, North Carolina.

Sugar cane tillers containing the immature leaf whorl used as source material for embryogenic cultures were collected and initiated into tissue culture. Transverse sections or leaf rolls of immature sugar cane leaf whorls were prepared essentially as described by Bower and Birch (1992). Leaf roll discs are generated by cutting 0.5-1.5 mm transverse sections. These cuts were made starting just above the apical meristem and up the shoot through to ˜2-3 cm above the apical meristem. Leaf roll discs were cultured on SC-D culture medium (4.3 g/L MS basal salts, 5 ml/L of 200×B5 vitamins, 30 g/L sucrose, 2 mg/L 2,4-D, adjust pH to 5.8 with 1 N KOH, and 7 g/L Phytoblend). Each plate contained 6-8 leaf rolls and plates were placed in a plastic culture box and transported to dark culture room at 28° C. Leaf rolls were cultured for about 14 days.

The responding leaf roll discs were broken up and spread out on SC-D fresh media plates. They were then placed in a dark room at 28° C. for an additional 14-18 days. During this time, embryogenic cultures developed on the culture plates.

### Example 2

### Pre-Culture Treatment Prior to Transformation

Pre-culture treatments can be performed in one of two ways. In the first way, the embryogenic cultures on the first plates are allowed to grow for 7-28 days prior to Agrobacterium inoculation. The best quality embryogenic tissue pieces are then selected for transformation. In the second method, the cultures on the first plates are allowed to grow for 7-18 days. Then the best quality embryogenic cultures are selected and placed onto fresh plates and placed in the dark room at 28° C. for an additional 7-40 days. Finally, the best quality embryogenic cultures are selected for transformation.

### Example 3

### Transformation of Embryo Genic Tissues

Although the following describes Agrobacterium-mediated transformation, this invention is not limited by the method of transformation. Methods for sugar cane transformation are known in the art, including for example U.S. Pat. No. 8,742,202 and U.S. patent application Ser. No. 13/378,497, each of these references incorporated herein by reference in its entirety. For this example, binary vectors in Agrobacterium tumefaciens strain such as LB4404 or EHA101 were used for transformation. A small amount of the Agrobacteria from a vial stored in −80° C. was taken with a sterile disposable plastic inoculating loop and placed on a plate. Agrobacteria was spread with the loop or cell spreader, to create a thin layer of cells over the surface of the growth media. Plates were placed in the 28° C. incubator for ˜2 days prior to use. Agrobacterium cells were collected from the plate using a sterile disposable plastic inoculation loop and suspended in liquid infection medium, for example SCInoc (modified from Khanna, et al. 2004) in a sterile disposable plastic tube. The tube was vortexed until Agrobacterium cells were uniformly dispersed in the suspension. Light absorption of the bacterial suspension was measured in a spectrophotometer and diluted to A₆₆₀ of 0.1-0.85. Acetosyringone was added to a final concentration of 40-80 mg/L (200-400 μM) to induce virulence gene expression.

The selected cultures were immediately infected with Agrobacterium by removing the culture from the media and mixing the isolated embryogenic cultures with the solution described above comprising the bacterial suspension. Various pretreatments may be applied to the target tissue in order to make the plant cells more amenable to gene delivery such as heat shock etc. The mixture was incubated for at least 1 minute or up to overnight at room temperature. Various treatments may be applied during inoculation to improve contact between bacteria and plant cells, such as sonication, vacuum infiltration etc. Following infection, the cultures were removed from the Agrobacterium suspension and moved to co-cultivation. The calli were then transferred to petri dishes. The dishes were sealed with plastic film for co-cultivation in the dark at 22° C. for 2 to 3 days. After co-cultivation, embryogenic cultures were transferred to recovery medium without selection agent such as SCRecov (4.3 g/L MS basal salts, 5 ml/L of 200×B5 vitamins, 30 g/L sucrose, 1-3 mg/L 2,4-D, 250 mg/L Ticaracillin, adjust pH to 5.8 with 1 N KOH, and 7 g/L Phytoblend) with appropriate antibiotics to inhibit Agrobacterium growth. The recovery plates with the explants were incubated for 2-10 days at 28° C. in the dark. After the recovery period, the explants were transferred to pre-regeneration selection media, for example SCM (4.3 g/L MS basal salts, 5 ml/L of 200×B5 vitamins, 20 g/L sucrose, 1-3 mg/L 2,4-D, 3 g/L mannose, 250 mg/L Ticaracillin, adjust pH to 5.8 with 1 N KOH, and 7 g/L Phytoblend) (with appropriate antibiotics) and were cultured at 28° C. in the dark for 3 weeks. After 3-5 weeks in pre-regeneration/selection media any proliferating sectors were selectively sub-cultured to regeneration/selection media, for example SCManRegen (4.3 g/L MS basal salts, 5 ml/L of 200×B5 vitamins, 24 g/L sucrose, 2 mg/L BA, 3 g/L mannose, adjust pH to 5.8 with 1 N KOH, and 7 g/L Phytoblend) with appropriate antibiotics for regeneration induction. These were cultured at 28° C. in the dark for 1 week. The tissue was then moved onto Regeneration induction media, for example SCR (4.3 g/L MS basal salts, 5 ml/L of 200×B5 vitamins, 20 g/L sucrose, 0.5 mg·L NAA, adjust pH to 5.8 with 1 N KOH, and 7 g/L Phytoblend) cultured in the light at 28° C. with 16 hr/day light. After 2 weeks, developing shoots were transferred to plant containers for shoot elongation and rooting.

### Example 4

### Transient Expression Affected by Length of Pre-Culture Treatment

Embryogenic cultures were generated as described in Example 1. Cultures were allowed to grow on plates containing solid media for 7-18 days. Then the best quality embryogenic tissues were selected and placed onto fresh SC-D media plates and placed in the dark room at 28° C. for an additional 4-14 days as the pre-culture treatment described as the second option in Example 2. Finally, the best quality embryogenic cultures were selected for Agrobacterium-mediated transformation and transformed using binary vector 19634, which comprises nucleotide sequence encoding the phosphomannose isomerase selectable marker and nucleotide sequence comprising an expression cassette encoding the cyan fluorescent protein (CFP). Following transformation, the embryogenic cultures were cultured on Recovery media for 6 days. After the 6 days, the embryogenic cultures were scored for transformed cells by screening for clusters, or spots, of CFP-expressing tissue on each embryogenic culture using a Leica MZFLIII microscope (Vashaw Scientific, Inc). The results are shown in Table 1.

TABLE 1
Transient CFP expression in US commercial
sugar cane variety No. 1

| Pre-culture | % of pieces of | # of spots per piece |
|---|---|---|
| Treatment | embryogenic culture | of embryogenic |
| (days) | with CFP spots | culture |
| 4 | 20 | <20 |
| 7 | 50 | 20-50 |
| 14 | 90 | >50 |

Surprisingly, a longer pre-culture treatment results in a higher percentage of embryogenic cultures transiently expressing CFP following transformation. In theory, this suggests that the pre-culture treatment of the embryogenic tissue allows for more efficient initial transgene delivery to the target cells, which are actively dividing cells within each piece of embryogenic tissue.

### Example 5

### Transformation Efficiency Affected by Pre-Culture Treatment

Although the length of the pre-culture treatment increased transient expression of the reporter gene CFP, stable transformation efficiencies for sugar cane are poorly predicted by transient expression. To determine transformation efficiency, embryogenic cultures of US commercial sugar cane variety No. 1 prepared by pre-culture treatments of various lengths of time were transformed with binary vectors comprising a nucleotide sequence encoding for the phosphomannose isomerase selectable marker using Agrobacterium-mediated transformation. The embryogenic cultures were co-cultivated, allowed to recover, and then transformed tissue was selected, developing shoots were generated, and finally shoots were transferred to plant containers as described in Example 3. Events were confirmed by TaqMan assay, using methods well-known in the art, which detected the presence of the T-DNA delivered by the Agrobacteria. Table 2 shows the number of events generated per starting gram of embryogenic culture using three different pre-culture treatments. Results are presented on fresh weight basis as the best option for comparison across genotypes that vary in culture morphology.

TABLE 2
Transformation efficiency on glyphosate selection
in US commercial sugar cane variety No. 1

| Pre-culture | Starting | | |
|---|---|---|---|
| Treatment | Fresh | # of | |
| (days) | Weight (g) | Events | Events/g |
| 4 | 5.22 | 3 | 0.57 |
| 7 | 5.66 | 6 | 1.06 |
| 14 | 5.45 | 18 | 3.3 |

These results indicate that, surprisingly, increasing the length of the pre-culture treatment results in an increase in transformation efficiency.

### Example 6

### Transformation Efficiency Affected by Pre-Culture Treatment in Multiple Sugar Cane Varieties and Selectable Marker/Selection Systems

Sugar cane is known to be recalcitrant to transformation. Additionally, it is known that a method of transformation which has some level of success with one variety of sugar cane may not necessarily work with a different variety. Therefore, different sugar cane varieties were tested to determine if the pre-culture treatment increased transformation efficiency for multiple varieties. Embryogenic cultures from Brazil commercial sugar cane varieties No. 1 and No. 2 were prepared, transformed, and transformed plants were regenerated using the methods described above. Tables 3 and 4 show the number of events generated per starting gram of embryogenic culture using different pre-culture treatments.

TABLE 3
Transformation efficiency on glyphosate selection
in Brazil commercial sugar cane variety No. 1

| Pre-culture | Starting | | |
|---|---|---|---|
| Treatment | Fresh | # of | |
| (days) | Weight (g) | Events | Events/g |
| 7 | 3.41 | 2 | 0.59 |
| 14 | 3.86 | 13 | 3.37 |
| 21 | 4.65 | 53 | 11.40 |
| 28 | 3.2 | 10 | 3.13 |
| 35 | 3.2 | 9 | 2.81 |

TABLE 4
Transformation efficiency on glyphosate selection
in Brazil commercial sugar cane Variety No. 2

| Pre-culture | Starting | | |
|---|---|---|---|
| Treatment | Fresh | # of | |
| (days) | Weight (g) | Events | Events/g |
| 7 | 5 | 1 | 0.20 |
| 15 | 10 | 19 | 1.90 |
| 21 | 5 | 15 | 3.00 |
| 30 | 5 | 2 | 0.40 |

Tables 3 and 4 show that the length of the pre-culture treatment affects the transformation efficiency of both Brazil Commercial varieties 1 and 2. These surprising results indicate that the pre-culture treatment increases transformation efficiency in more than one sugar cane variety.

Although this pre-treatment increases transformation efficiency in multiple sugar cane varieties when glyphosate selection is used, it is possible that the observed effect is related to the selection method used, rather than the pre-culture treatment. To determine if this is so, Brazil commercial sugar cane variety No. 1 and 2 were also tested using a different vector, which encoded the selectable marker phosphomannose isomerase. Embryogenic cultures from Brazil commercial sugar cane varieties 1 and US commercial sugar cane variety No. 1 were prepared, transformed, and transformed plants were regenerated using the methods described above. Tables 5 and 6 show the number of events generated per starting gram of embryogenic culture using different pre-culture treatments.

TABLE 5
Transformation efficiency on mannose selection
in Brazil commercial sugar cane variety No. 1

| Pre-culture | Starting | | |
|---|---|---|---|
| Treatment | Fresh | # of | |
| (days) | Weight (g) | Events | Events/g |
| 7 | 3.41 | 8 | 2.35 |
| 14 | 3.86 | 43 | 11.14 |
| 21 | 4.65 | 142 | 30.54 |
| 28 | 3.2 | 43 | 13.44 |
| 35 | 3.2 | 42 | 13.13 |

TABLE 6
Transformation efficiency on mannose selection
in US commercial sugar cane variety No. 1

| Pre-culture | Starting | | |
|---|---|---|---|
| Treatment | Fresh | # of | |
| (days) | Weight (g) | Events | Events/g |
| 14 | 4 | 39 | 9.75 |
| 21 | 6 | 95 | 15.83 |

Tables 5 and 6 show that the length of the pre-culture treatment affects the transformation efficiency under mannose selection with 2 different varieties. These surprising results indicate that the pre-culture treatment increases transformation efficiency using more than one selectable marker and selection system.

Although the foregoing invention has been described in some detail by way of illustration and example for purposes of clarity of understanding, it will be obvious that certain changes and modifications may be practiced within the scope of the appended claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

What is claimed is:

1. An improved method for preparing sugar cane embryogenic cultures prior to transformation, comprising the steps of:
a) excising a segment of plant tissue from a shoot of sugar cane;
b) culturing said segment of plant tissue to produce sugar cane embryogenic tissues; and
c) performing a pre-culture treatment comprising sub-culturing responding sugar cane embryogenic tissues to fresh media and culturing for a period of time of at least 7 days on the same media,
wherein sugar cane embryogenic tissues cultured according to step (c) are transformable.

2. The method of claim 1, wherein the period of time in step (c) is 7 to 60 days.

3. The method of claim 2, wherein the period of time in step (c) is 7 to 30 days.

4. The method of claim 1, followed by transformation mediated by Agrobacterium.

5. The method of claim 4, wherein the sugar cane embryogenic cultures are removed from the media of step (c) and mixed with a solution comprising Agrobacterium as part of the transformation process.

6. An improved method of preparing sugar cane embryogenic cultures for transformation, comprising the steps of:
a) excising a segment of plant tissue from a shoot of sugar cane;
b) culturing said segment of plant tissue to produce sugar cane embryogenic cultures;
c) sub-culturing responding sugar cane embryogenic cultures on fresh media;
d) optionally repeating step (c) after at least 7 days, and
e) performing a pre-culture treatment comprising sub-culturing responding sugar cane embryogenic tissues to fresh media and culturing for a period of time of at least 7 days on the same media,
wherein sugar cane embryogenic tissues cultured according to step (e) are transformable.

7. The method of claim 6, wherein said period of time in step (e) is 7 to 60 days.

8. The method of claim 6, wherein said period of time in step (e) is 7 to 30 days.

9. The method of claim 6, followed by transformation mediated by Agrobacterium.

10. The method of claim 9, wherein the sugar cane embryogenic cultures are removed from the media of step (e) and mixed with a solution comprising Agrobacteria as part of the transformation process.

12. A transgenic sugar cane tissue, plant part, or plant produced by the method of claim 1.

13. A transgenic sugar cane tissue, plant part, or plant produced by the method of claim 6.
